(19) United States
(12) Patent Application Publication
(10) Pub. No.: US 2009/0105689 A1
(43) Pub. Date: Apr. 23, 2009

(54) Removable push-off tab for IV catheter

(76) Inventor: Mark V. Mitchum, Panama City, FL (US)

Correspondence Address: LITMAN LAW OFFICES, LTD., POST OFFICE BOX 15035, CRYSTAL CITY STATION, ARLINGTON, VA, 22215-0035, US

(21) Appl. No.: 12/071,358

(22) Filed: Feb. 20, 2008

Related U.S. Application Data
(60) Provisional application No. 60/960,951, filed on Oct. 22, 2007.

(51) Int. Cl. A61M 25/01

(52) U.S. Cl. 604/523

(57) ABSTRACT

The removable push-off tab for an IV catheter is adapted for releasable attachment to a hub of an intravenous catheter assembly. The removable push-off tab is adapted for bearing against a push-off plate to provide an increased bearing surface for pushing the catheter into the patient's vein. The removable push-off tab has opposed concave and convex faces and an opening formed substantially centrally therethrough. The opening is dimensioned and configured for releasable mounting on the hub of the catheter.

### CROSS-REFERENCE TO RELATED APPLICATION

This application claims the benefit of U.S. Provisional Patent Application Ser. No. 60/960,951, filed Oct. 22, 2007.

### BACKGROUND OF THE INVENTION

1. Field of the Invention

The present invention relates to medical devices for performing intravenous (IV) catheterization, and particularly to a removable push-off tab for an intravenous catheter that eases the task of inserting the catheter into a vein.

2. Description of the Related Art

FIG. 2 shows a conventional assembly 14 for insertion of an IV catheter. The catheter itself generally includes a hub 18 and a cannula 20 extending from the hub 18. A needle 21 is inserted through the lumen defined by the cannula 20 so that the pointed tip of the needle 21 extends just beyond the end of the catheter. The needle assembly includes a flashback chamber to collect any blood that spurts back through the cannula 20 into the hub 18. The hub 18 includes a push-off plate 16 extending from the side of the hub 18.

In use, the needle 21 is inserted into the catheter as shown in FIG. 2. A tourniquet is placed proximal to the venipuncture site, and the tip of the needle 21 is directed to pierce the vein. The catheter is pushed down the needle 21 through the puncture in the skin and vein until blood appears in the flashback chamber, indicating that the vein has been pierced. The tip of the cannula 20 should generally extend about ¼″ into the vein when the catheter is properly inserted. Many catheters include the push-off plate 16 on the side of the hub 18 to assist in the process of pushing the catheter into the vein.

However, such conventional push-off plates 16 are typically too small to provide adequate leverage to push the catheter off the needle 21 and into the vein. It would be desirable to provide an add-on gripping member that provides increased leverage for proper insertion of the catheter. Thus, a removable push-off tab for an IV catheter solving the aforementioned problems is desired.

### SUMMARY OF THE INVENTION

The removable push-off tab for an IV catheter is adapted for releasable attachment to a hub of an intravenous catheter assembly above the push-off plate. The removable push-off tab is adapted for placing one's finger against the tab to push the catheter into the vein, providing a larger bearing surface than the conventional push-off plate.

The removable push-off tab is a releasable tab having opposed concave and convex faces. The releasable tab also has an opening formed substantially centrally therethrough. The opening is dimensioned and configured for releasably mounting the tab on the hub of the intravenous catheter assembly to bear against the push-off plate. When mounted on the catheter, the convex face is positioned so that it faces the cannula, and the concave face is in an opposed position; i.e., facing the flanges on the catheter hub, the tab abutting the push-off plate to provide a pushing surface for the user's finger(s).

These and other features of the present invention will become readily apparent upon further review of the following specification and drawings.

### BRIEF DESCRIPTION OF THE DRAWINGS

FIG. 1 is an environmental, perspective view of a removable push-off tab for an IV catheter according to the present invention.

FIG. 2 is a perspective view of a prior art IV catheter assembly.

FIG. 3 is an environmental perspective view of the removable push-off tab for an IV catheter according to the present invention, shown exploded from a catheter and needle assembly.

FIG. 4 is a front view of the removable push-off tab for an IV catheter according to the present invention.

FIG. 5 is a rear view of the removable push-off tab for an IV catheter according to the present invention.

FIG. 6 is a bottom view of the removable push-off tab for an IV catheter according to the present invention.

Similar reference characters denote corresponding features consistently throughout the attached drawings.

### DETAILED DESCRIPTION OF THE PREFERRED EMBODIMENTS

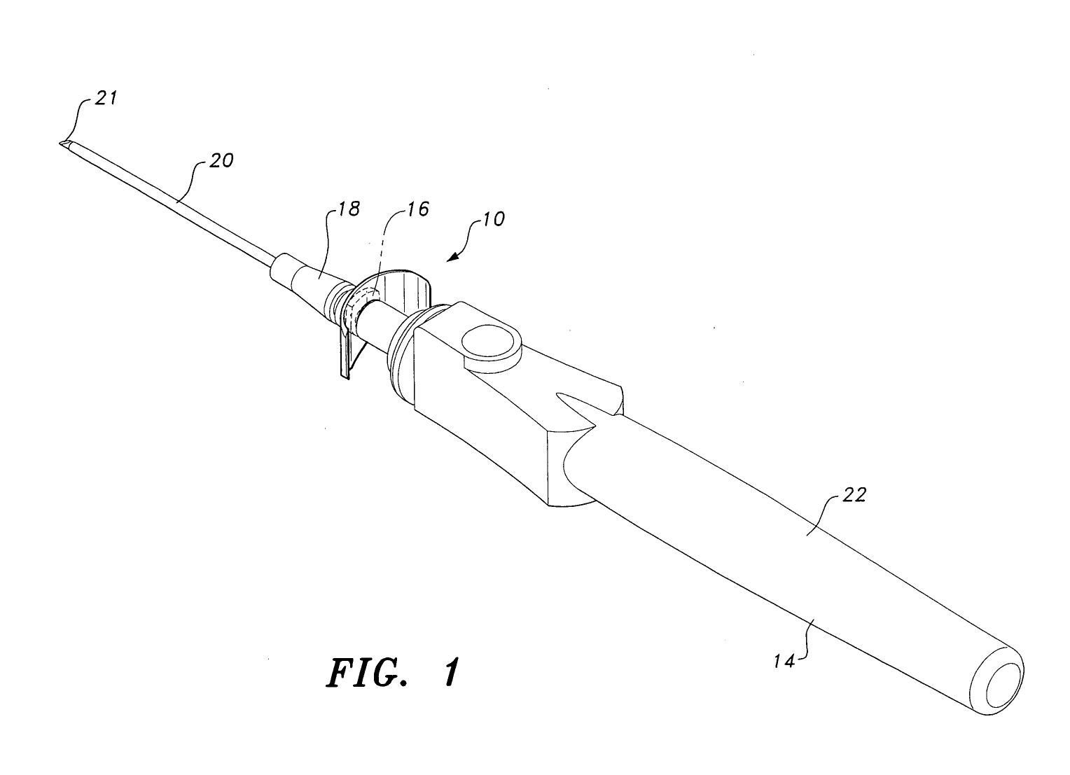

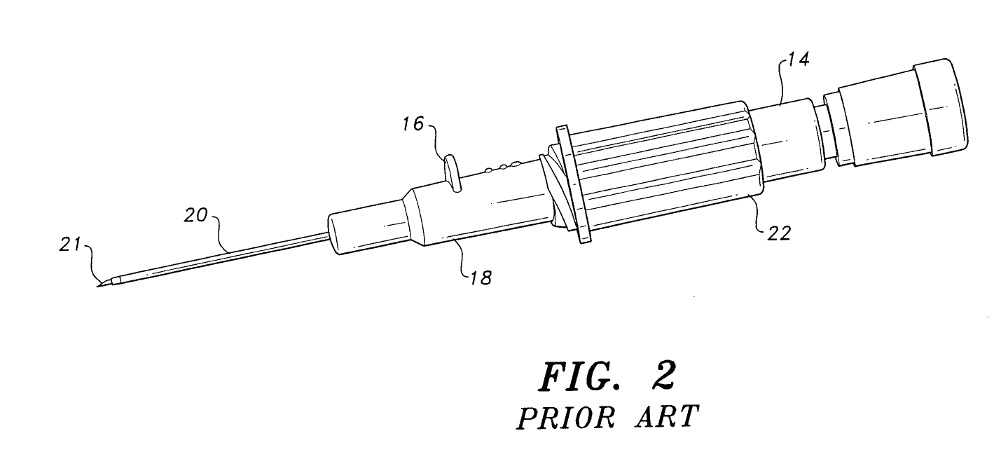

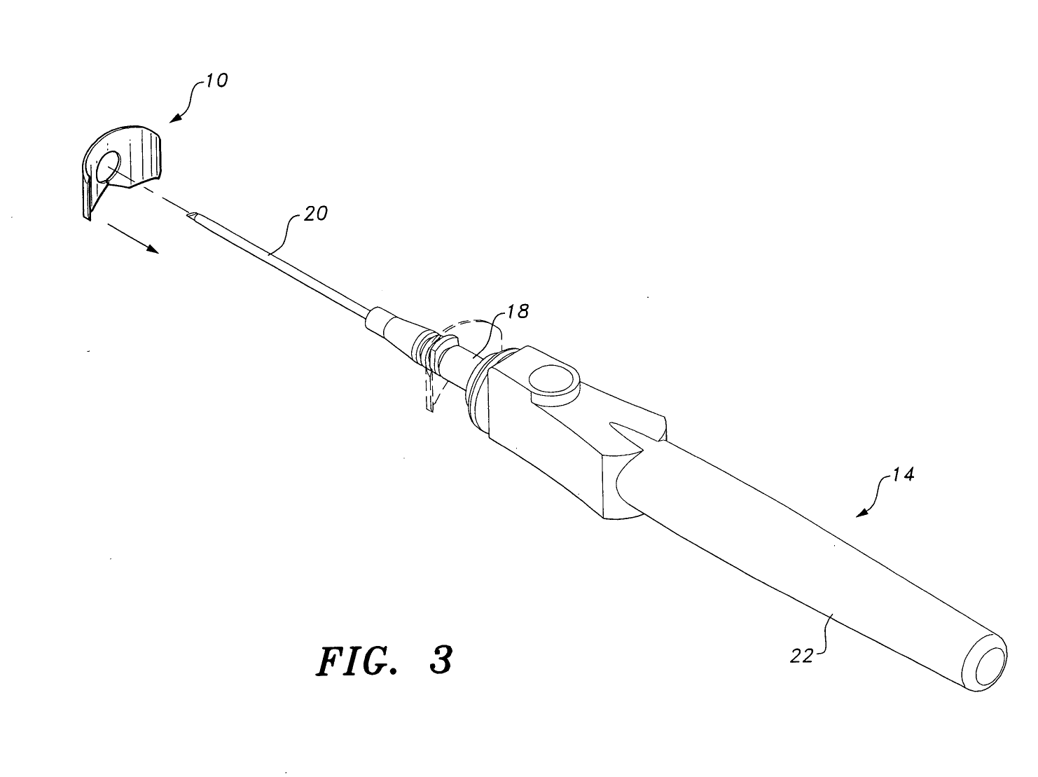

The present invention is directed towards a removable push-off tab for an IV catheter. As shown in FIG. 1, the removable push-off tab 10 is a gripping member, which is adapted for releasable attachment to a hub 18 of an intravenous catheter assembly 14. The removable push-off tab 10 is adapted for grasping by a user in order to facilitate the insertion of a needle associated with the catheter assembly into a patient. The removable push-off tab 10 is particularly useful for insertion by a user using only one hand to insert the needle; i.e., the push-off tab 10 is grasped by the fingers of only one hand during insertion. In FIG. 1, the push-off tab 10 is shown mounted on hub 18, adjacent push-off plate 16 (which is integrally formed on hub 18, as described above with reference to the prior art catheter of FIG. 2). Catheter assembly 14 is shown for exemplary purposes only, and it should be understood that the removable push-off tab 10 may be used with catheter assemblies either already having an integral push-off plate 16, or catheter assemblies which do not include such a push-off plate.

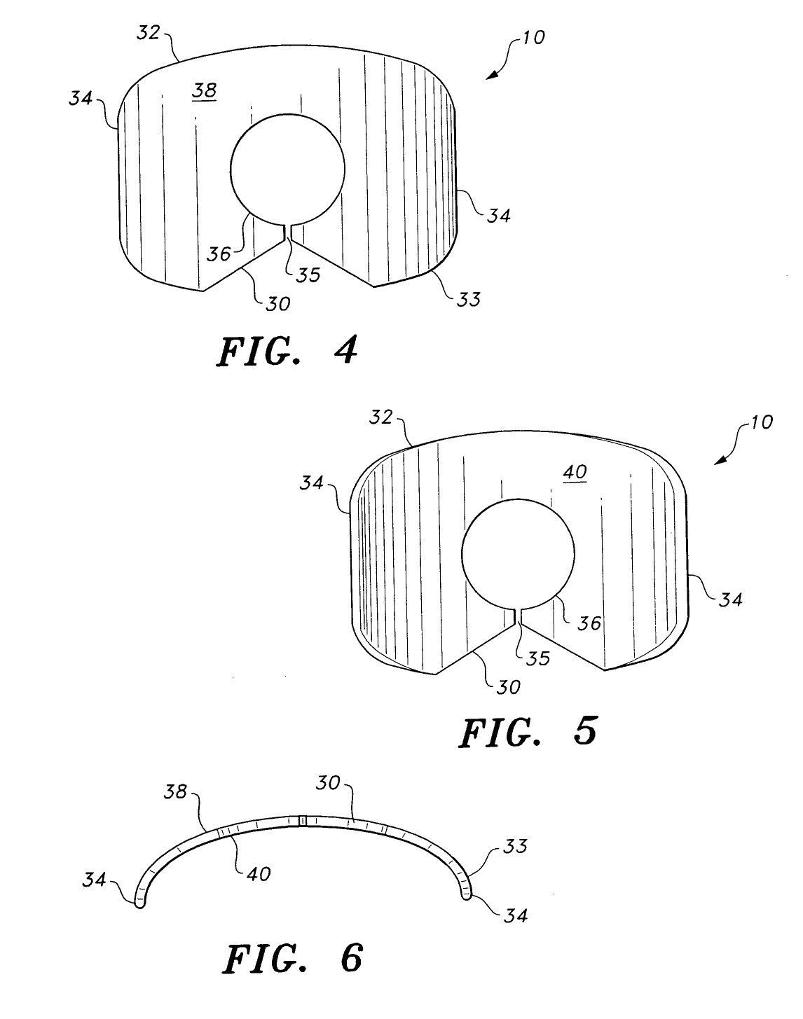

The removable push-off tab 10 is a releasable tab having opposed concave and convex faces 40, 38, with the releasable tab 10 having an opening 36 formed substantially centrally therethrough. The opening 36 is sized and contoured such that the releasable tab 10 may be releasably mounted on the hub 18 of the intravenous catheter assembly 14, with the hub 18 being received within the opening 36. In FIGS. 4 and 5, the opening 36 is shown as having a circular contour with particular dimensions. It should be understood that the sizing and contouring of opening 36 is dependent upon the particular IV assembly the tab 10 is being used with.

When mounted on the catheter assembly 14, the convex face 38 is positioned facing the cannula 20 and needle 21 of the catheter assembly 14, and the concave face 40 is in an opposed position; i.e., facing a flashback chamber 22 (generally associated with conventional IV assemblies) of the IV assembly 14. As shown, for a catheter assembly 14 having a push-off plate 16, the convex face 38 is positioned against the push-off plate 16, thus providing a concave surface facing the user such that the user's fingers may be received within the concave sides of concave surface 40 during pushing of hub 18 when inserting needle 21 into a patient.

The releasable tab 10 includes a pair of laterally opposed side edges 34, which are adapted for grasping by the user. Preferably, each side edge 34 has a substantially arcuate contour (as best shown in FIGS. 4 and 5). Further, the releasable tab 10 has opposed upper and lower edges 32, 33, with the upper edge 32 having a substantially continuous arcuate contour. The lower edge 33 also preferably has a substantially arcuate contour, however, in the preferred embodiment, a substantially V-shaped recess 30 is formed substantially centrally in the lower edge 33. V-shaped recess 30 is provided to easily guide and connect opening 36 about the hub 18.

As shown, a slit 35 is preferably formed between opening 36 and the vertex of V-shaped recess 30. Thus, the circumference of opening 36 is adjustable, allowing for secure and stable mounting about hubs 18 of varying size. Further, the tab 10 is preferably formed from a relatively flexible material, such as plastic, allowing for adjustment on a variety of hubs 18, and further allowing for resilient bending and gripping of the side edges 34 during usage.

As described above, the convex face 38 of tab 10 is preferably positioned adjacent the push-off plate 16, thus increasing the gripping surface area for the user's fingers, and providing an ergonomic, concave receiving surface for the user's fingers. The concave-convex curvature of tab 10 is provided to ergonomically mold the user's index fingertip upon pushing and advancement of needle 21. Further, with the user's index fingertip positioned on the flexing, curved tab 10, the pushing inwardly by the user's index finger will serve to stabilize the catheter, thus preventing premature separation thereof from the needle 21.

It should be understood that tab 10 may be dimensioned and configured as desired. As shown, the tab 10 is preferably sized to not only increase the vertical gripping surface (compared to the integral push-off plate of the prior art IV catheter), but also to extend the horizontal gripping surface. Further, in the preferred embodiment, lower edge 33, upper edge 32, and side edges 34 all have substantially rounded contours in order to provide comfortable and secure gripping surfaces for the user's fingers. Similarly, the corners of the tab 10 are also rounded, for the user's comfort and for enhancing grippability.

It is to be understood that the present invention is not limited to the embodiments described above, but encompasses any and all embodiments within the scope of the following claims.

I claim:

1. A removable push-off tab for an IV catheter, comprising a releasable tab having opposed concave and convex faces, the releasable tab having an opening formed substantially centrally therethrough, the opening being dimensioned and configured for releasably mounting on a hub of an intravenous catheter assembly bearing against the catheter push-off plate in order to provide an increased bearing surface for pushing the catheter into a patient's vein.

2. The removable push-off tab for an IV catheter as recited in claim 1, wherein the releasable tab is substantially arcuate in cross section.

3. The removable push-off tab for an IV catheter as recited in claim 1, wherein the opening is substantially circular.

4. The removable push-off tab for an IV catheter as recited in claim 1, wherein the releasable tab defines an upper edge, the upper edge being substantially convex.

5. The removable push-off tab for an IV catheter as recited in claim 4, wherein the releasable tab defines a pair of laterally opposed side edges, each side edge being substantially convex.

6. The removable push-off tab for an IV catheter as recited in claim 5, wherein the releasable tab defines a lower edge, the lower edge having a pair of laterally opposed end portions and a central portion.

7. The removable push-off tab for an IV catheter as recited in claim 6, wherein each of the laterally opposed end portions of the lower edge is substantially convex.

8. The removable push-off tab for an IV catheter as recited in claim 7, wherein a pair of lower intersections between the pair of laterally opposed end portions of the lower edge and the pair of laterally opposed side edges are rounded.

9. The removable push-off tab for an IV catheter as recited in claim 8, wherein a pair of upper intersections between the upper edge and the pair of laterally opposed side edges are rounded.

10. The removable push-off tab for an IV catheter as recited in claim 7, wherein a substantially V-shaped recess is formed in the central portion of the lower edge, the substantially V-shaped recess defining a vertex, the vertex being positioned adjacent the opening.

11. The removable push-off tab for an IV catheter as recited in claim 10, wherein a slot is formed through the releasable tab, the slot extending between the opening and the vertex of the substantially V-shaped recess.

12. The removable push-off tab for an IV catheter as recited in claim 1, wherein the releasable tab is formed from a flexible material.

13. The removable push-off tab for an IV catheter as recited in claim 12, wherein the releasable tab is formed from plastic.
